(19) United States
(12) Patent Application Publication
(10) Pub. No.: US 2024/0115213 A1
(43) Pub. Date: Apr. 11, 2024

(54) DIAGNOSING AND TRACKING STROKE WITH SENSOR-BASED ASSESSMENTS OF NEUROLOGICAL DEFICITS

(71) Applicant: The Regents of the University of California, Oakland, CA (US)

(72) Inventors: Vishwajith Ramesh, San Diego, CA (US); Nadir Weibel, San Diego, CA (US); Gert Cauwenberghs, San Diego, CA (US); Brett C. Meyer, San Diego, CA (US); Kunal Agrawal, San Diego, CA (US)

(21) Appl. No.: 18/263,941

(22) PCT Filed: Feb. 5, 2022
(86) PCT No.: PCT/US22/15385

§ 371 Date: Aug. 2, 2023

Related U.S. Application Data
(60) Provisional application No. 63/146,450, filed on Feb. 5, 2021.

(51) Int. Cl. A61B 5/00; A61B 5/0205; G16H 50/30

(52) U.S. Cl. CPC A61B 5/7282; A61B 5/0205; A61B 5/4064; A61B 5/7267; A61B 5/7445; A61B 5/746; G16H 50/30; A61B 3/14

(57) ABSTRACT

A method, a system, and a computer program product for detecting and/or determining occurrence of a neurological event in a subject. Data corresponding to one or more symptoms, detected by one or more sensors, associated with a subject is received. The sensors include sensors positioned directly on the subject and/or sensors positioned away from the subject. One or more symptom values are assigned to one or more detected symptoms. A severity score for each of the symptoms is determined. The severity scores are determined using one or more machine learning models receiving the assigned symptom values as input. A prediction that the subject is experiencing at least one neurological event and at least a type of the neurological event is generated using a combination of the determined severity scores corresponding to the symptoms. A generation of one or more alerts is triggered based on the prediction. One or more user interfaces are generated for displaying the alerts.

### CROSS-REFERENCE TO RELATED APPLICATIONS

The present application claims priority to U.S. Provisional Patent Appl. No. 63/146,450 to Ramesh et al., filed Feb. 5, 2021, and entitled “Diagnosing and Tracking Stroke with Sensor-Based Assessments of Neurological Deficits”, and incorporates its disclosure herein by reference in its entirety.

### TECHNICAL FIELD

The current subject matter generally relates to data processing, and in particular, detecting and/or diagnosing various neurological conditions/events, including stroke.

### BACKGROUND

Stroke is a major leading cause of death and disability in the US. The only therapy for stroke is utilized in less than 5% of acute strokes however because it has to be administered within 3 hours of the onset of symptoms. Accurately diagnosing a stroke as soon as possible after the onset of symptoms is tough as it requires a subjective evaluation by a stroke specialist in a hospital. Moreover, stroke outcome prediction is currently crude, and stroke deficit scales are generally unable to predict if a patient will do well or very poorly.

### SUMMARY

In some implementations, the current subject matter relates to a computer implemented method for detecting and/or determining occurrence of a neurological event in a subject. The method may include receiving data corresponding to one or more symptoms, detected by one or more sensors, associated with a subject. The sensors may include sensors positioned directly on the subject and/or sensors positioned away from the subject. One or more symptom values may then be assigned to one or more detected symptoms. A severity score for each of the symptoms may be determined. The severity scores may be determined using one or more machine learning models receiving the assigned symptom values as input. A prediction that the subject may be experiencing at least one neurological event and at least a type of the neurological event may be generated using a combination of the determined severity scores corresponding to the symptoms. A generation of one or more alerts may be triggered based on the prediction. One or more user interfaces may be generated for displaying the alerts.

In some implementations, the current subject matter may be configured to include one or more of the following optional features. The neurological event may include a stroke. The sensors may include at least one of the following: an audio sensor, a video sensor, a biological sensor, a medical sensor, and any combination thereof.

In some implementations, the symptoms may include at least one of the following: one or more neurological symptoms, one or more biological parameters, one or more symptoms determined based on one or more physiological responses from the subject, and any combination thereof. The physiological responses may include at least one of the following: one or more eye movements, one or more facial landmarks, one or more body joint positions, one or more pupil movements, one or more speech patterns, and any combination thereof. The symptoms may include at least one of the following: dysarthria, aphasia, facial paralysis, gaze deficit, nystagmus, body joint weakness, hemiparesis, ataxia, dyssynergia, dysmetria, and any combination thereof. The biological parameters may include at least one of the following: an electrocardiogram, an electroencephalogram, a blood pressure, a pulse, and any combination thereof.

In some implementations, the type of the neurological event may include at least one of the following: an acute stroke, an ischemic stroke, a hemorrhagic stroke, a transient ischemic attack, a warning stroke, a mini-stroke, and any combination thereof.

In some implementations, the receiving may include at least one of the following: passively receiving the data without requiring the subject to perform an action, receiving the data resulting from actively requiring the subject to perform an action, manually entering the data, querying stored data, and any combination thereof.

In some implementations, the method may also include continuously monitoring the subject using the sensors, determining, based on the continuous monitoring, one or more new symptom values, updating the determined severity score for each of the symptoms, and the generated prediction, triggering a generation of one or more updated alerts based on the updated prediction, and generating one or more updated user interfaces for displaying the updated alerts.

In some implementations, at least one of the receiving, the assigning, the determining, the generating the prediction, the triggering, and the generating the one or more user interfaces is performed in substantially real time.

In some implementations, the generating of the user interfaces may include arranging one or more graphical objects corresponding to the symptoms, the prediction, the alerts, in the user interfaces in a predetermined order.

Implementations of the current subject matter can include systems and methods consistent including one or more features are described as well as articles that comprise a tangibly embodied machine-readable medium operable to cause one or more machines (e.g., computers, etc.) to result in operations described herein. Similarly, computer systems are also described that may include one or more processors and one or more memories coupled to the one or more processors. A memory, which can include a computer-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to optical edge detection, it should be readily understood that such features are not intended to be limiting. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 illustrates an exemplary system for detecting and/or determining occurrence of a neurological condition/event (e.g., a stroke), according to some implementations of the current subject matter;

FIG. 2 illustrates an exemplary system for detecting and/or determining occurrence of a neurological condition (e.g., a stroke), according to some implementations of the current subject matter;

FIG. 3a illustrates an exemplary user interface that may be generated using one or more user interface components shown in FIG. 1, according to some implementations of the current subject matter;

FIG. 3b illustrates an exemplary user interface that may be generated using one or more user interface components shown in FIG. 1, according to some implementations of the current subject matter;

FIG. 4 illustrates an exemplary system, according to some implementations of the current subject matter; and

FIG. 5 illustrates an exemplary method, according to some implementations of the current subject matter.

### DETAILED DESCRIPTION

One or more implementations of the current subject matter relate to methods, systems, articles of manufacture, and the like that may, among other possible advantages, provide an ability to detect and determine occurrence of a neurological condition/event (e.g., a stroke) in a subject.

The current standard for stroke assessment is a series of motor and cognitive tests done by an experienced clinician, the National Institute of Health Stroke Scale (NIHSS). The NIHSS is a neurological examination assessing consciousness, eye movements, visual field, motor and sensory impairments, ataxia, speech, cognition, and inattention. Stroke evaluation using the NIHSS is performed by a skilled neurologist, which limits its application to specific clinical situations (i.e. when neurologists can be physically present). In fact, during emergencies in the field, diagnosis of stroke by emergency medical services (EMS) is frequently incorrect. Moreover, evaluation during stroke using the NIHSS has elements with poor reliability between examiners. Patients suffering from stroke mimic (e.g. multiple sclerosis or intracranial tumor) may have symptoms that overlap with those of stroke. These patients may therefore also score high on the NIHSS test and be misclassified: 21% of patients treated for stroke based, in part, on the results of NIHSS were later found to have no stroke-associated brain infarcts with follow-up imaging. This inaccuracy is in part because the NIHSS is not a comprehensive test, nor is it specific for the diagnosis of stroke.

Magnetic resonance imaging (MRI) is often used in conjunction with the NIHSS to identify a stroke but it's cumbersome to use, expensive, and takes time. An MRI cannot often be done within the 3 hour time window of the single FDA-approved treatment for stroke, an anti-clotting agent known as tPA. The NIHSS is a somewhat of a quick and dirty diagnostic assessment, done in place of an MRI. It is also very difficult to fit an MRI in an ambulance for diagnosis of a stroke soon after its incidence, although specific ambulances incorporating an MRI do exist in small numbers.

While other conventional systems have explored the use of electroencephalography to diagnose stroke, such studies involve cumbersome and expensive research headsets and are not designed to be deployed in the acute settings in which we are interested.

Acute stroke diagnosis presents several challenges that motivate the development and use of computational aids. Stroke diagnosis typically requires a series of motor and cognitive tests designed to quickly and quantitatively assess impairments, such as the National Institute of Health Stroke Scale. Stroke evaluation by a clinician or first responder is subjective and contingent on their prior experience with stroke assessments, especially since some stroke symptoms are hard to discern. Moreover, potential stroke patients are often evaluated in time-sensitive emergency environments that encourage missed or misdiagnosed strokes. To provide decision support to clinicians and/or first responders in a real-time or near real-time way, there is a need for a method that leverages computational techniques for identifying symptoms of neurological conditions/events, e.g., a stroke. In some implementations, the current subject matter relates to system(s), method(s), and/or article(s) of manufacture for identifying the presence and/or absence of stroke symptoms through contactless sensing to quicken the recognition of stroke and help reduce errors, is such an aid.

In some exemplary implementations, subjects with suspected ischemic strokes may be recorded using various sensors, e.g., a video camera, audio microphone (e.g., in a smartphone, a tablet, etc. (“sensing device”)). Subjects may be recorded passively and/or at rest and/or while a clinician and/or first responder conducts a neurological examination, a series of motor and cognitive tests. The sensor data processing unit may be configured to function to extract biological features that may be used by the stroke symptom detector from video and audio feeds. The sensor data processing unit may include a set of sensor data extraction processes for each type of biological feature required by the stroke symptom detector. For example, a speech process may produce representations of a subject's vocal patterns from raw speech; this type of biological feature is used by the stroke symptom detector to identify the presence or absence of dysarthria or slurred speech, a stroke symptom. Similarly, the face process may extract relevant keypoints in a subject's face from video for facial paralysis detection. The sensor data processing unit may also include a pupil process that may track the subject's pupil and eye movements, a body joint process that may track the spatial coordinates of the subject's body joints, and a finger tracking process that may monitor the movements of the hands and fingers, and/or any other processes. The sensor data processing unit may be wholly and/or partially instantiated in a remote cloud hosted platform and/or on the sensing device.

In some implementations, the stroke symptom detector may be a suite of signal processing and machine learning algorithms that may be trained to detect the presence or absence of acute stroke symptoms such as dysarthria, aphasia, facial paralysis, gaze deficit, nystagmus, body joint weakness, hemiparesis, ataxia, dyssynergia, dysmetria, and/or any combination thereof. The severity of each symptom may also be scored by the stroke symptom detector. Furthermore, the stroke symptom detector functions to predict the likelihood of the most probably types of stroke in a subject, based on the symptoms detected. As with the sensor data processing unit, the stroke symptom detector may be wholly and/or partially located in a remote cloud hosted platform and/or on the sensing device. Since the extraction of biological features from sensor data and the use of such features to detect stroke symptoms with machine learning are both susceptible to inaccuracies in different scenarios, there may be a cross talk between the two components to minimize errors. For example, if poor lighting results in an inaccurate detection of facial paralysis by the stroke symptom detector, the face process may be modified to accommodate for those environmental conditions.

In some implementations, the current subject matter's monitor system may function to inform an entity (clinician or first responder) of the status of a monitored subject. The monitor system may display one or more predictions of the stroke symptom detector (e.g., the presence of a symptom and its severity on a color scale) and may additionally receive live and/or capture video, images, and/or audio from the sensing device. Biological features extracted by the sensor data processing unit may also be displayed on the monitor system, either directly or via abstracted representations. The monitor system may be an application and/or website accessible by a smartphone or tablet that can be the same as the sensing device. The monitor system may additionally include a user interface for accepting user (e.g., a clinician, a medical professional, etc.) input, such as the scores from a neurological examination.

Moreover, in some exemplary, non-limiting implementations, the current subject matter process(es) may be configured to provide automatic detection of stroke symptoms of one or more subjects in a real-time and/or substantially real-time simply from an audio, a video, and/or any other sensory data. The subjects may be passively observed without requiring them to perform some action and/or hold some position. The current subject matter may also determine and/or display a likelihood of a particular type of stroke based on symptoms that were detected and/or analyzed. Further, the current subject matter may be configured to provide a stroke signature for an individual, an easily interpretable indicator of the overall severity of stroke symptoms over time.

In some implementations, the current subject matter may be configured to have one or more of the following advantages and/or applications. As one of the exemplary applications, the current subject matter may be applied as a clinical decision support system for acute stroke diagnosis in emergency departments, such as to facilitate the diagnosis of stroke by emergency medicine physicians in the absence of expert neurologists. As another exemplary application, the current subject matter may be used for a field diagnosis. For example, first responders and/or emergency crews may use the current subject matter system to automatically detect stroke symptoms of an individual in a field and inform triage. As yet another exemplary application, the current subject matter system may be used in various medical and/or non-medical facilities (e.g., nursing homes, hospitals, clinics, medical offices, elderly care facilities, homes etc.) to provide a individuals and/or clinicians a tool for remote monitoring of stroke symptoms in at-risk individuals. As yet a further exemplary application, the current subject matter system may be used to track the severity of symptoms in rehabilitating stroke subjects over time.

In some implementations, the current subject matter may be configured to include one or more machine learning and/or signal processing pipelines to analyze data acquired through one or more sensors, video cameras, image cameras, audio sensors, depth cameras as well as other technology medical technology, e.g., EEG headsets, etc. in a symptom-specific way. For example, the current subject matter system may be configured to track pupil saccades and/or nystagmus events for the purposes of performing computer vision analysis and/or signal processing. To identify hemiparesis, the current subject matter system may be configured to use machine learning. Further, a user interface may be generated to display predictions/assessments generated by the current subject matter. Additionally, inputs submitted by users (e.g., medical personnel, clinicians, etc.) relating to own assessments may be accepted by the system and used to perform further analysis in determining occurrence of a stroke. The current subject matter may be designed to aid in stroke diagnosis in acute emergency settings as well as assessments performed in rehabilitation settings (e.g., tracking patient symptoms over time).

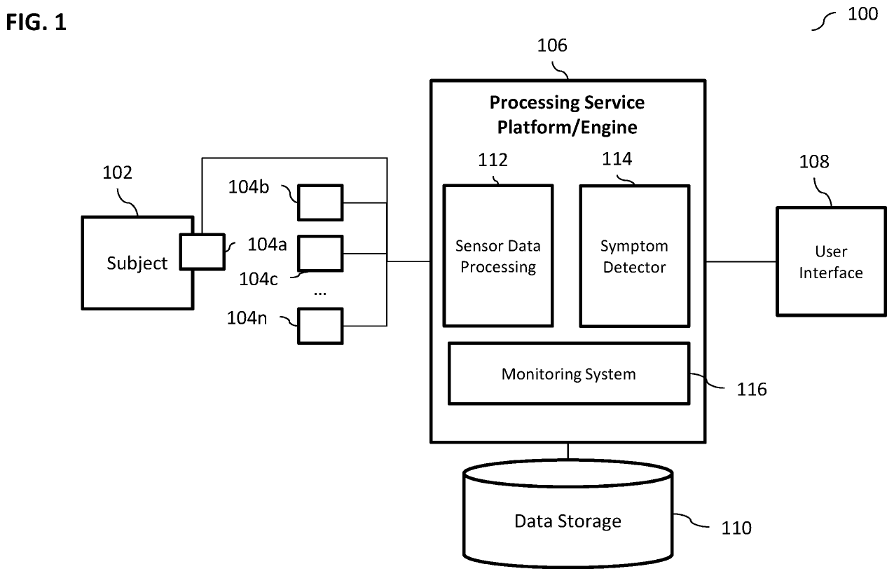

FIG. 1 illustrates an exemplary system 100 for detecting and/or determining occurrence of a neurological condition/event (e.g., a stroke), according to some implementations of the current subject matter. The system 100 may include one or more sensor devices 104 (a, b, c, . . . n) that may be used to monitor, sense and/or observe a subject 102 and/or detect various symptoms associated with the subject 102. The system 100 may also include a processing service platform and/or engine 106, a user interface 108, and a data storage 110. The system 100 may be configured to operate in one or more cloud computing environments.

The engine 106 may include one or more computing elements (which may, for example, as discussed below, include one or more processors, one or more servers, one or more computing engines, one or more memory and/or storage locations, one or more databases, etc.) such as, sensor data processing component 112, a symptom detector component 114, and a monitoring system 116. The system 100 may be configured to provide an end user (e.g., a medical professional, a lay person, etc.) with an indication of whether the subject 102 is or is not experiencing a neurological condition, e.g., a stroke. Such indication may be based on an analysis of data received from the sensors, as will be discussed below.

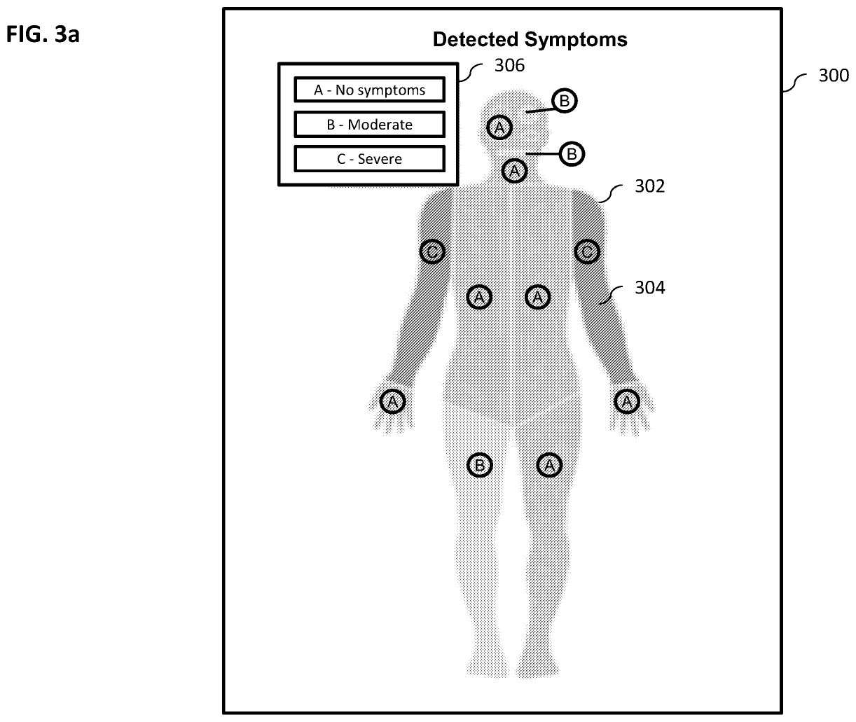

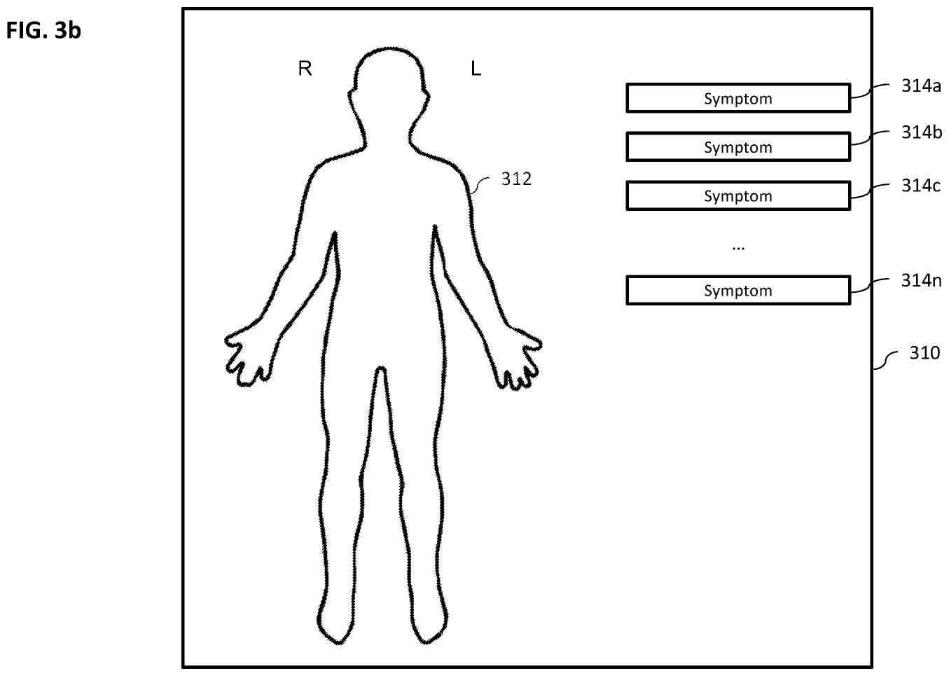

The processing service platform/engine 106 may include a processor, a memory, and/or any combination of hardware/software, and may be configured to analyze data obtained from the sensors 104 to determine whether the subject 102 is or is not experiencing a neurological condition. The engine 106 may be configured to include one or more processing and/or machine learning pipelines and/or implement one or more machine learning models to determine whether the subject 102 is or is not experiencing a neurological condition. The engine 106 may be configured to cause generation of various alerts and/or indications (e.g., as shown in FIGS. 3a-b) relating to whether the subject 102 may be experiencing such neurological condition. The alerts/indications may be graphically displayed using the user interface component 108. Additionally, any obtained data and/or results of evaluation may be stored in a storage location and/or a data storage 110. A computing component (e.g., component 104-116) may refer to a piece of software code that may be configured to perform a particular function, a piece and/or a set of data (e.g., data unique to a particular subject and/or data available to a plurality of subjects) and/or configuration data used to create, modify, etc. one or more software functionalities to a particular user and/or a set of users. The engine 106 may include one or more artificial intelligence and/or learning capabilities that may rely on and/or use various data, e.g., data related to and/or identifying one or more symptoms and/or parameters associated with the subject 102 that have been currently obtained (e.g., as a result of monitoring, detecting, etc. by sensors 104), previously obtained (e.g., by sensors 104, and/or determined by the engine 106) and/or generated by the engine 106.

In some implementations, the data that may be received and/or processed by the engine 106 may include any data, metadata, structured content data, unstructured content data, embedded data, nested data, hard disk data, memory card data, cellular telephone memory data, smartphone memory data, main memory images and/or data, forensic containers, zip files, files, memory images, and/or any other data/information. The input and/or the output data (as generated by the engine 106) may be in various formats, such as text, numerical, alpha-numerical, hierarchically arranged data, table data, email messages, text files, video, audio, graphics, etc. One or more of the above data may be collected in real-time, continuously, during predetermined periods of time, periodically (e.g., at certain preset periods of time, e.g., every 30 seconds, every 5 minutes, every hour, etc.). The data may be queried upon execution of a certain feature of the current subject matter process.

The system 100 may be configured to include one or more servers, one or more databases, a cloud storage location, a memory, a file system, a file sharing platform, a streaming system platform and/or device, and/or in any other platform, device, system, etc., and/or any combination thereof. One or more components of the system 100 may be communicatively coupled using one or more communications networks. The communications networks can include at least one of the following: a wired network, a wireless network, a metropolitan area network (“MAN”), a local area network (“LAN”), a wide area network (“WAN”), a virtual local area network (“VLAN”), an internet, an extranet, an intranet, and/or any other type of network and/or any combination thereof.

The components of the system 100 may include any combination of hardware and/or software. In some implementations, such components may be disposed on one or more computing devices, such as, server(s), database(s), personal computer(s), laptop(s), cellular telephone(s), smartphone(s), tablet computer(s), and/or any other computing devices and/or any combination thereof. In some implementations, these components may be disposed on a single computing device and/or can be part of a single communications network. Alternatively, or in addition to, the components may be separately located from one another.

Referring back to FIG. 1, one or more sensors 104 may be configured to be positioned directly on the subject 102 (e.g., a patient at a medical facility and/or any other individual being observed by the system 100). The directly positioned sensors 104 may include leads that may be attached to the subject 102 to detect and/or monitor various physiological, neurological, biological, movement, health, and/or other parameters of and/or associated with the subject 102, which may be indicative of various symptoms that may the subject 102 may be exhibiting and/or experiencing. The sensors 104 may also be positioned remotely from the subject 102. The remotely positioned sensors 104 may include one or more video, audio, graphics, textual, etc. capturing and/or video, audio, graphics, textual, etc. capable devices (e.g., cameras, smartphone devices, tablet devices, etc.). Such devices may be configured to take a video of the subject 102 and/or record subject 102's speech. The sensors 104 may be configured to passively and/or actively monitor, observe, and/or detect physiological, neurological, biological, movement, health, and/or other parameters of and/or associated with the subject 102.

Alternatively, or in addition to, various data may be supplied to the engine 106, for instance, through the user interface 108 and/or queried from the data storage 110. The data may include, but is not limited to subject's personal data (e.g., name, gender, address, etc.), various health data (e.g., weight, age, medical conditions, cholesterol levels, etc.), one or more biological parameters (e.g., an electrocardiogram, an electroencephalogram, a blood pressure, pulse, etc.) and/or any other data, and/or any combination thereof), etc. In some implementations, the data may be queried by the engine 104 from the data storage 110 and/or one or more third party databases. The engine 104 may determine which database may contain requisite information and then connect with that database to execute a query and retrieve appropriate information. In some implementations, the engine 106 can include various application programming interfaces (APIs) and/or communication interfaces that may allow interfacing with other components of the system 100.

In some implementations, the engine 106 may be configured to receive and process, using sensor data processing component 112, the data (e.g., either from sensors 104, user interface 108, and/or data storage 110) and perform an assessment of whether the subject 102 may or may not be experiencing a neurological condition, e.g., a stroke. The engine 106 may be configured to apply one or more separate and/or common machine learning models to each type of input data that it receives to determine symptoms that are being experienced by the subject 102 and/or determine severity (e.g., by determining a severity score) of each such symptom. The engine 106 may be configured to use a symptom detector component 114 that may be configured to store such machine learning models and/or perform various machine learning and/or artificial intelligence processes.

In some exemplary implementations, the engine 106, using component 114, may be configured to distinguish between different types of data (e.g., based on a type of sensor 104 that is supplying data to the engine 106, e.g., audio, video, etc.) to ascertain and/or analyze various symptoms experienced by the subject 102. For example, data received from an audio sensor 104 may be used to determine and/or analyze one or more speech patterns of the subject. The engine 106 may invoke a machine learning model that may be trained specifically to analyze speech (e.g., through analysis of natural language processing, audio levels, clarity of speech, etc.) to determine whether the subject 102 is exhibiting, for example, dysarthria, aphasia, and/or any other speech-related symptoms/conditions.

Data received from a video sensor 104 that may be focused on the subject's face may be configured to determine and/or analyze one or more facial landmarks (e.g., cheeks, mouth, etc.). This data may be used by the engine 106 to determine whether the subject 102 is exhibiting a facial paralysis and/or any other related symptoms/conditions. Such determination by the engine 106 may implement another machine learning model that may be specifically trained for facial paralysis recognition.

Further, data received from the same or another video sensor 104 (e.g., a sensor 104 focused specifically on the eyes of the subject 102) may be used to determine and/or analyze one or more of eye and/or pupil movement of the subject 102. The engine 106 may use yet another machine learning model that may be trained to recognize eye and/or pupil movements. Using this model, the engine 106 may be configured to determine that the subject 102 may be experiencing gaze deficits, nystagmus, and/or any other symptoms/conditions.

Moreover, yet another sensor may be configured to monitor (e.g., through video, audio, and/or in any other way) body joint positions and/or movements of the subject 102. For example, such body joints may include, but are not limited to, fingers, shoulders, elbows, knees, head, spine, etc. The engine 106 may be configured to determine (e.g., through using yet another machine learning model trained to analyze positions and/or movements of body joints) whether the subject 102 is exhibiting weakness (e.g., including hemiparesis), ataxia (e.g., including dyssynergia, dysmetria, etc.), and/or any other body joint symptoms/conditions.

In some implementations, the engine 106 may be configured to use a single machine learning model and/or multiple machine learning models that may be trained using a single and/or different training data sets relating to each of the above symptoms/conditions. Each of the detected symptoms may be configured to be associated and/or assigned a particular symptom value that may be compared against a particular threshold value to determine whether the determined symptom values exceeds a corresponding threshold. If so, the engine 106 may be configured to display (e.g., on the user interface 108) an appropriate indication. The engine 106, using one or more machine learning models that use assigned symptom values as input, may also determine and display (e.g., on the user interface 108) a severity score and/or an indication of each such symptom/condition (e.g., through use of various color and/or letter indications and/or other alerts).

The engine 106 may then be configured to use the severity scores and/or indications associated with each symptom to generate a prediction that the subject 102 may or may not be experiencing at least one neurological disorder (e.g., a stroke). Additionally, using one or more and/or a combination of the severity scores/indications associated with each experienced symptom, the engine 106 may be configured to determine a type of neurological disorder being experienced by the subject 102. For example, the engine 106 may be configured to determine that the subject 102 is experiencing an acute stroke, an ischemic stroke, a hemorrhagic stroke, a transient ischemic attack, a warning stroke, a mini-stroke, and any combination thereof.

Once the engine 106 determines that the subject 102 may or may not be experiencing a neurological condition (e.g., a stroke), the engine 106's monitoring system 116 may be configured to trigger generation of one or more alerts based on the above prediction. The alerts may be displayed on the user interface 108. The alerts may include a visual, an audio, a graphical, and/or any other indicators. The alerts may be specific to a particular part of the subject 102 (e.g., a body part, a physiological parameter (e.g., blood pressure, pulse, etc.)) that may be exhibiting above normal (e.g., exceeding a corresponding threshold value) values. The alert may be an overall alert that may be indicative of the subject experiencing a neurological condition, e.g., a stroke. The engine 106 may be configured to cause a specific graphical arrangement of the alerts and/or any other indicators on the user interface 108 (e.g., as shown in FIGS. 3a-b).

In some implementations, the alerts may be transmitted to one or more systems (e.g., hospitals, clinics, first responders, etc.) for evaluation and/or subsequent action. In some implementations, the system 100 and/or any of the processes performed by any of its components may be configured to operate in real time and/or substantially in real-time.

In some implementations, the system 100 may be configured to perform continuous monitoring of the subject 102. The monitoring (including obtaining new data from sensors 104 and/or being entered by a user (e.g., medical professional, clinician, home user, etc.) of the system 100) may be performed during predetermined periods of time and/or for a predetermined period of time. By way of a non-limiting example, monitoring may be performed for 30 seconds at a time during a period of 10 minutes (and/or during any other periods of time and/or frequency of monitoring). This may allow the system 100 to determine whether the subject 102 is truly experiencing symptoms indicative of a particular neurological condition and/or whether some determined symptoms may been in error.

Moreover, any such monitoring may be performed passively and/or actively. Passive monitoring of the subject 102 may include observing the subject 102 without requiring the subject 102 to perform any specific actions (e.g., move arms, move head, blink an eye, speak a certain phrase, etc.). Active monitoring may require the subject 102 to perform specific actions.

The engine 106 may use any updated data/information obtained as a result of the continuous monitoring of the subject 102 to determine one or more new values associated with one or more symptoms and/or conditions. Such new values may be used to update severity scores associated with one or more symptoms and trigger generation of any updated alerts. Any data that may be obtained by the system 100, including severity scores, values associated with the various symptoms/conditions, etc. may be stored in the data storage 110.

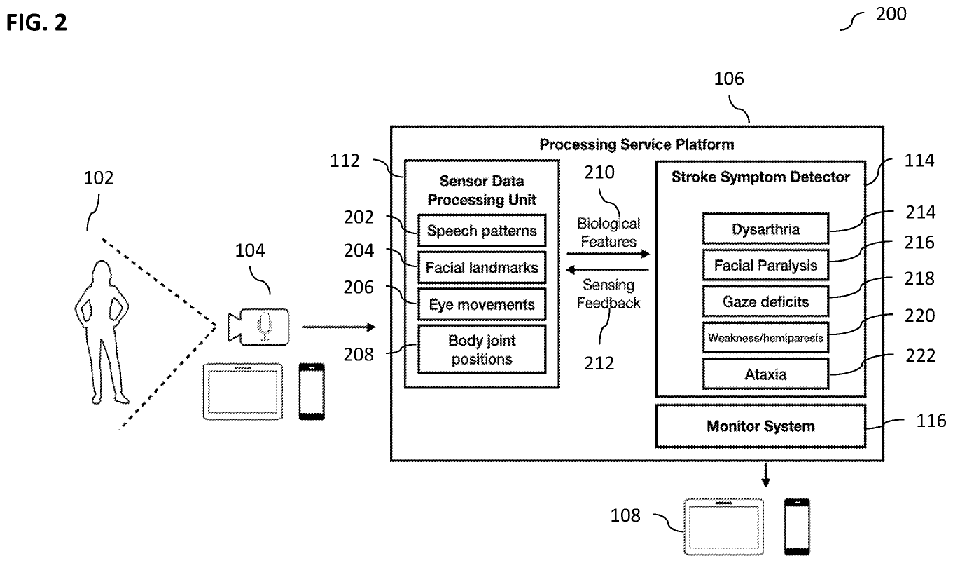

FIG. 2 illustrates an exemplary system 200 for detecting and/or determining occurrence of a neurological condition (e.g., a stroke), according to some implementations of the current subject matter. The system 200 may be similar to the system 100 and may include one or more sensors 104, the processing service platform/engine 106, and one or more user interface devices 108. The system 200 may also include a database (e.g., data storage 110) that may be used for storage of various data.

Similar to FIG. 1, the engine 106 may include a sensor data processing unit or component 112, a stroke symptom detector component 114 and a monitoring system 116. The sensor data processing unit 112 may include one or more components 202-208 configured to process and/or analyze, using one or more machine learning models and/or other processing components/pipelines, various sensor data that may be received from the sensors 104 that monitor, observe, etc. the subject 102. Each such machine learning model may be specific to a particular data that is being sensed and may be appropriately invoked, such as, for example, using an identifier associated with the received sensor data (e.g., extracted from a data packet containing sensor data as received from the sensor 104), for processing the sensor data. Alternatively, or in addition to, a single machine learning model may be used to process all sensor data. By way of a non-limiting example, the components 202-208 may include a speech patterns component 202, a facial landmarks component 204, an eye movements component 206, a body joints positions component 208, as well as any others. Moreover, the components 202-208 may include one or more components that may be configured to process and/or analyze data related to various biological parameters of the subject 102 (e.g., EEG, ECG, blood pressure, pulse, etc.). The components 202-208 may also process and/or analyze data that may be manually entered, such as, using one or more user interface devices 108. Such data may include data that may be entered by a user of the system 100 (e.g., a doctor, a medical professional, a home user, etc.) that may be observing the subject 102 and assessing various conditions of the subject 102. As can be understood a single component (rather than multiple components 202-208) may be used to process sensor data.

Upon processing the received sensor data, the components 202-208 may be configured to extract one or more feature values (e.g., biological, neurological, etc.) 210 associated with the received data and provide such feature values 210 (e.g., in a form of a vector) to the stroke symptom detector component 114. The component 114 may be configured to include one or more components 214-222 configured to process and/or analyze, using one or more machine learning models and/or other processing components/pipelines, the feature values 210 to ascertain presence of a particular symptom. Similarly to the above, each such machine learning model may be specific to a particular feature value vector received from the component 112 and, likewise, may be invoked for further processing and/or analysis, such as, to determine presence of a particular symptom and/or determine severity of such symptom (such as, for example, through comparison of the values to one or more corresponding thresholds). Alternatively, or in addition to, a single machine learning model may be used to process all feature vectors. By way of a non-limiting example, the components 214-222 may include a dysarthria component 214 configured to process feature vector values from the speech patterns component 202; a facial paralysis component 216 configured to process feature vector values from the facial landmarks component 204; a gaze deficits component 218 configured to process feature vector values from the eye movement component 206; and the weakness/hemiparesis component 220 and ataxia component 222 may be configured to process feature vector values from the body joint positions component 208. As can be understood, the above components 214-222 are exemplary and any other components and/or a single component may be used to process such feature vector values 210. The components 214-222 may be configured to provide one or more sensing feedback 212 to the sensor data processing component 112. The feedback 212 may be used to adjust operation of the sensors 104 and/or processing of the component 112.

FIG. 3a illustrates an exemplary user interface 300 that may be generated using one or more user interface components 108 shown in FIG. 1, according to some implementations of the current subject matter. The user interface 300 may be configured to include an outline 302 of a human body (alternatively or in addition to, the outline 302 may be an image of the subject 102). The outline 302 may be divided into several parts 304, where each part may correspond to, for example, arms, hands, legs, torso, head, eyes, mouth, ears, cheeks, etc. The parts 304 may also distinguish between right and left sides of the body, as those may be useful in determining a type of neurological condition that the subject 102 may be experiencing.

The user interface 300 may also include a legend 306 that may be used for identifying specific severities associated with each symptom being experienced by a particular part of the body. The severity in the legend 306 may include, for example, “A”—no symptoms, “B”—moderate severity symptoms, and “C”—high or severe symptoms. Alternatively or in addition to, color designations may be used to illustrate severity (e.g., green—no symptoms, yellow—moderate severity; and red—high severity).

Once the engine 106 (as shown in FIG. 1) performs analysis of the data received from the sensors 104, the engine 106 may be configured to display the severities of the symptoms that are being experienced by the parts of the body. For example, as shown in FIG. 3a, both arms are showing high severity (“C”) symptoms (e.g., the subject 102 cannot move the arms), while hands are showing no symptoms (e.g., the subject 102 can move the hands). Further, the subject 102 may also exhibiting moderate severity (“B”) symptoms in the right leg. Additionally, moderate severity symptoms are also being experienced by the subject's left eye and mouth. Based on these indications, the system 100 (shown in FIG. 1) may be configured to determine that the person is experiencing a neurological condition or an event, e.g., a stroke. Additionally, based on the locations of the observed symptoms, the system 100 may be configured to determine a type of the stroke that is being experienced by the subject 102. As stated above, the system 100 may then display an appropriate alert indicating a particular neurological condition experienced by the subject 102 and that the subject 102 may require an immediate medical attention.

FIG. 3b illustrates an exemplary user interface 310 that may be generated using one or more user interface components 108 shown in FIG. 1, according to some implementations of the current subject matter. The user interface 310 may be interactive and may be used by a user (e.g., a medical professional, a first responder, etc. (not shown in FIG. 3b)) that may be observing the subject 102, to enter user's observations of the subject's symptoms, status, etc. The user interface 310 may also include an outline 312 of a human body (alternatively or in addition to, the outline 312 may be an image of the subject 102). Similar to outline 302, the outline 312 may be divided into several parts, where each part may correspond to, for example, arms, hands, legs, torso, head, eyes, mouth, ears, cheeks, etc. The outline may also distinguish between right (“R”) and left (“L”) sides of the body.

The user interface 310 may also include one or more drop down menus 314 (a, b, c, . . . , n) that may be used by the user to select a specific part of the body in the outline 312 and enter and/or select symptoms that the user observed. The data entered by the user may be combined with the data obtained by the sensors 104 (as shown in FIG. 1) and may be used by the engine 106 (not shown in FIG. 3b) to ascertain symptoms of the subject 102 and determine whether the subject is experiencing a neurological condition and/or event.

As stated above, some of the advantages of the system 100, as shown in FIGS. 1-3b, may include an ability to observe the subject 102 and determine whether the subject is or is not experiencing a particular neurological condition and/or event, such as, a stroke. Such observations may be passive, whereby the subject is not required to perform any specific activities. The current subject matter system may further be used by an untrained individual to determine whether the subject 102 is experiencing a particular neurological condition and/or event to allow such individual to quickly obtain qualified medical help. This may be helpful in a multitude of settings, e.g., homes, businesses, hospitals, clinics, elderly care facilities, public transportation, public spaces, etc.

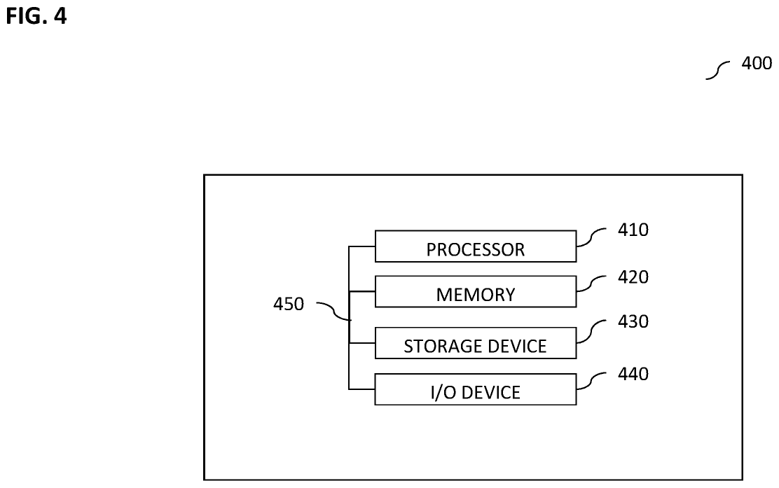

FIG. 4 depicts a block diagram illustrating a computing system 400 consistent with implementations of the current subject matter. For example, the system 400 can be used to implement the devices and/or system disclosed herein (e.g., host one or more aspect of FIG. 1). As shown in FIG. 4, the computing system 400 can include a processor 410, a memory 420, a storage device 430, and input/output devices 440. The processor 410, the memory 420, the storage device 430, and the input/output devices 440 can be interconnected via a system bus 450. The processor 410 is capable of processing instructions for execution within the computing system 400. Such executed instructions can implement one or more components of, for example, the trusted server, client devices (parties), and/or the like. In some implementations of the current subject matter, the processor 410 can be a single-threaded processor. Alternately, the processor 410 can be a multi-threaded processor. The processor may be a multi-core processor having a plurality or processors or a single core processor. The processor 410 is capable of processing instructions stored in the memory 420 and/or on the storage device 430 to display graphical information for a user interface provided via the input/output device 440.

The memory 420 is a computer readable medium such as volatile or non-volatile that stores information within the computing system 400. The memory 420 can store data structures representing configuration object databases, for example. The storage device 430 is capable of providing persistent storage for the computing system 400. The storage device 430 can be a floppy disk device, a hard disk device, an optical disk device, or a tape device, or other suitable persistent storage means. The input/output device 440 provides input/output operations for the computing system 400. In some implementations of the current subject matter, the input/output device 440 includes a keyboard and/or pointing device. In various implementations, the input/output device 440 includes a display unit for displaying graphical user interfaces.

According to some implementations of the current subject matter, the input/output device 440 can provide input/output operations for a network device. For example, the input/output device 440 can include Ethernet ports or other networking ports to communicate with one or more wired and/or wireless networks (e.g., a local area network (LAN), a wide area network (WAN), the Internet).

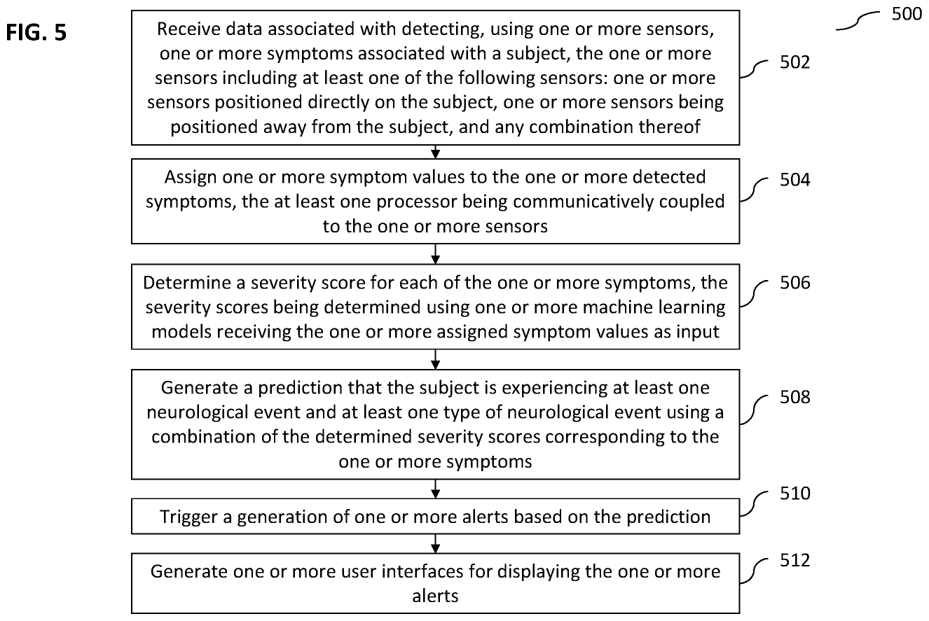

FIG. 5 illustrates an exemplary process 500 for detecting and/or determining occurrence of a neurological condition, disorder, event (“event”) in a subject, according to some implementations of the current subject matter. The process 500 may be configured to be performed by the system 100 and 200 shown in FIGS. 1 and 2, respectively. The process 500 may be configured to generate one or more user interfaces 300 and/or 310, as shown in FIGS. 3a and 3b, respectively. In particular, the engine 106, as shown in FIG. 1, may be configured to perform one or more operations of the process 500.

At 502, the engine 106 and/or any other processor may be configured to receive data corresponding to one or more symptoms, detected by one or more sensors 104, and associated with the subject 102. As shown in FIG. 1, the sensors 104 may include at least one of the following sensors: one or more sensors positioned directly on the subject, one or more sensors being positioned away from the subject, and any combination thereof.

At 504, the engine 106 may be configured to assign one or more symptom values to the detected symptoms. These may include one or more feature vector values 210 shown in FIG. 2. The engine 106 may be communicatively coupled to the sensors 104.

At 506, the engine 106 may be configured to determine a severity score for each of the symptoms. The severity scores may be determined using one or more machine learning models receiving the assigned symptom values as input. The determination of the severity of symptoms may be performed by one or more components 214-222 of the stroke symptom detector 114, as shown in FIG. 2.

At 508, the engine 106 may be configured to generate a prediction that the subject 102 may be experiencing at least one neurological condition, event, and/or disorder (“event”), e.g., a stroke. The engine 106 may also determine a type of the neurological event using a combination of the determined severity scores corresponding to the one or more symptoms. At 510, the engine 106 may be configured to trigger a generation one or more alerts based on the prediction (e.g., using a user interface device 108) and generate one or more user interfaces (e.g., 300, 310, as shown in FIGS. 3a, 3b) for displaying the alerts.

In some implementations, the current subject matter may be configured to include one or more of the following optional features. The neurological event may include a stroke. The sensors may include at least one of the following: an audio sensor, a video sensor, a biological sensor, a medical sensor, and any combination thereof.

In some implementations, the symptoms may include at least one of the following: one or more neurological symptoms, one or more biological parameters, one or more symptoms determined based on one or more physiological responses from the subject, and any combination thereof. The physiological responses may include at least one of the following: one or more eye movements, one or more facial landmarks, one or more body joint positions, one or more pupil movements, one or more speech patterns, and any combination thereof. The symptoms may include at least one of the following: dysarthria, aphasia, facial paralysis, gaze deficit, nystagmus, body joint weakness, hemiparesis, ataxia, dyssynergia, dysmetria, and any combination thereof. The biological parameters may include at least one of the following: an electrocardiogram, an electroencephalogram, a blood pressure, a pulse, and any combination thereof.

In some implementations, the type of the neurological event may include at least one of the following: an acute stroke, an ischemic stroke, a hemorrhagic stroke, a transient ischemic attack, a warning stroke, a mini-stroke, and any combination thereof.

In some implementations, the receiving may include at least one of the following: passively receiving the data without requiring the subject to perform an action, receiving the data resulting from actively requiring the subject to perform an action, manually entering the data, querying stored data, and any combination thereof.

In some implementations, the method may also include continuously monitoring the subject using the sensors, determining, based on the continuous monitoring, one or more new symptom values, updating the determined severity score for each of the symptoms, and the generated prediction, triggering a generation of one or more updated alerts based on the updated prediction, and generating one or more updated user interfaces for displaying the updated alerts.

In some implementations, at least one of the receiving, the assigning, the determining, the generating the prediction, the triggering, and the generating the one or more user interfaces is performed in substantially real time.

In some implementations, the generating of the user interfaces may include arranging one or more graphical objects corresponding to the symptoms, the prediction, the alerts, in the user interfaces in a predetermined order.

The systems and methods disclosed herein can be embodied in various forms including, for example, a data processor, such as a computer that also includes a database, digital electronic circuitry, firmware, software, or in combinations of them. Moreover, the above-noted features and other aspects and principles of the present disclosed implementations can be implemented in various environments. Such environments and related applications can be specially constructed for performing the various processes and operations according to the disclosed implementations or they can include a general-purpose computer or computing platform selectively activated or reconfigured by code to provide the necessary functionality. The processes disclosed herein are not inherently related to any particular computer, network, architecture, environment, or other apparatus, and can be implemented by a suitable combination of hardware, software, and/or firmware. For example, various general-purpose machines can be used with programs written in accordance with teachings of the disclosed implementations, or it can be more convenient to construct a specialized apparatus or system to perform the required methods and techniques.

These computer programs, which can also be referred to programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, the subject matter described herein can be implemented on a computer having a display device, such as for example a cathode ray tube (CRT), a liquid crystal display (LCD) monitor, a head-mounted display (HMD), a holographic display, etc. for displaying information to the user and a keyboard and a pointing device, such as for example a mouse or a trackball, by which the user can provide input to the computer. Other kinds of devices can be used to provide for interaction with a user as well. For example, feedback provided to the user can be any form of sensory feedback, such as for example visual feedback, auditory feedback, or tactile feedback; and input from the user can be received in any form, including, but not limited to, acoustic, speech, or tactile input.

The subject matter described herein can be implemented in a computing system that includes a back-end component, such as for example one or more data servers, or that includes a middleware component, such as for example one or more application servers, or that includes a front-end component, such as for example one or more client computers having a graphical user interface or a Web browser through which a user can interact with an implementation of the subject matter described herein, or any combination of such back-end, middleware, or front-end components. The components of the system can be interconnected by any form or medium of digital data communication, such as for example a communication network. Examples of communication networks include, but are not limited to, a local area network (“LAN”), a wide area network (“WAN”), and the Internet.

The computing system can include clients and servers. A client and server are generally, but not exclusively, remote from each other and typically interact through a communication network. The relationship of client and server arises by virtue of computer programs running on the respective computers and having a client-server relationship to each other.

Although ordinal numbers such as first, second, and the like can, in some situations, relate to an order; as used in this document ordinal numbers do not necessarily imply an order. For example, ordinal numbers can be merely used to distinguish one item from another. For example, to distinguish a first event from a second event, but need not imply any chronological ordering or a fixed reference system (such that a first event in one paragraph of the description can be different from a first event in another paragraph of the description).

The foregoing description is intended to illustrate but not to limit the scope of the invention, which is defined by the scope of the appended claims. Other implementations are within the scope of the following claims.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations can be within the scope of the following claims.

1. A computer-implemented method, comprising:
receiving, using at least one processor, data corresponding to one or more symptoms, detected by one or more sensors, associated with a subject, the one or more sensors including at least one of the following sensors: one or more sensors positioned directly on the subject, one or more sensors being positioned away from the subject, and any combination thereof, the at least one processor being communicatively coupled to the one or more sensors;
assigning, using the at least one processor, one or more symptom values to the one or more detected symptoms;
determining, using the at least one processor, a severity score for each of the one or more symptoms, the severity scores being determined using one or more machine learning models receiving the one or more assigned symptom values as input;
generating, using the at least one processor, a prediction that the subject is experiencing at least one neurological event and at least a type of the at least one neurological event using a combination of the determined severity scores corresponding to the one or more symptoms;
triggering, using the at least one processor, a generation of one or more alerts based on the prediction; and
generating, using the at least one processor, one or more user interfaces for displaying the one or more alerts.

2. The method according to claim 1, wherein the at least one neurological event includes a stroke.

3. The method according to claim 1, wherein the one or more sensors include at least one of the following: an audio sensor, a video sensor, a biological sensor, a medical sensor, and any combination thereof.

4. The method according to claim 1, wherein the one or more symptoms include at least one of the following: one or more neurological symptoms, one or more biological parameters, one or more symptoms determined based on one or more physiological responses from the subject, and any combination thereof.

5. The method according to claim 4, wherein the one or more physiological responses include at least one of the following: one or more eye movements, one or more facial landmarks, one or more body joint positions, one or more pupil movements, one or more speech patterns, and any combination thereof.

6. The method according to claim 4, wherein the one or more symptoms include at least one of the following: dysarthria, aphasia, facial paralysis, gaze deficit, nystagmus, body joint weakness, hemiparesis, ataxia, dyssynergia, dysmetria, and any combination thereof.

7. The method according to claim 4, wherein the one or more biological parameters include at least one of the following: an electrocardiogram, an electroencephalogram, a blood pressure, a pulse, and any combination thereof.

8. The method according to claim 1, wherein the type of the at least one neurological event includes at least one of the following: an acute stroke, an ischemic stroke, a hemorrhagic stroke, a transient ischemic attack, a warning stroke, a mini-stroke, and any combination thereof.

9. The method according to claim 1 wherein the receiving includes at least one of the following: passively receiving the data without requiring the subject to perform an action, receiving the data resulting from actively requiring the subject to perform an action, manually entering the data, querying stored data, and any combination thereof.

10. The method according to claim 1, further comprising
continuously monitoring the subject using the one or more sensors;
determining, based on the continuous monitoring, one or more new symptom values;
updating, using the at least one processor, the determined severity score for each of the one or more symptoms, and the generated prediction;
triggering, using the at least one processor, a generation of one or more updated alerts based on the updated prediction; and
generating, using the at least one processor, one or more updated user interfaces for displaying the one or more updated alerts.

11. The method according to claim 1, wherein at least one of the receiving, the assigning, the determining, the generating the prediction, the triggering, and the generating the one or more user interfaces is performed in substantially real time.

12. The method according to claim 1, wherein the generating the one or more user interfaces includes arranging one or more graphical objects corresponding to the one or more symptoms, the prediction, the one or more alerts, in the one or more user interfaces in a predetermined order.

13. A system comprising:
at least one programmable processor; and
a non-transitory machine-readable medium storing instructions that, when executed by the at least one programmable processor, cause the at least one programmable processor to perform operations comprising:
receiving data corresponding to one or more symptoms, detected by one or more sensors, associated with a subject, the one or more sensors including at least one of the following sensors: one or more sensors positioned directly on the subject, one or more sensors being positioned away from the subject, and any combination thereof, the at least one programmable processor being communicatively coupled to the one or more sensors;
assigning one or more symptom values to the one or more detected symptoms;
determining a severity score for each of the one or more symptoms, the severity scores being determined using one or more machine learning models receiving the one or more assigned symptom values as input;
generating a prediction that the subject is experiencing at least one neurological event and at least a type of the at least one neurological event using a combination of the determined severity scores corresponding to the one or more symptoms;
triggering a generation of one or more alerts based on the prediction; and
generating one or more user interfaces for displaying the one or more alerts.

14. The system according to claim 13, wherein the at least one neurological event includes a stroke.

15. The system according to claim 13, wherein the one or more sensors include at least one of the following: an audio sensor, a video sensor, a biological sensor, a medical sensor, and any combination thereof.

16. The system according to claim 13, wherein the one or more symptoms include at least one of the following: one or more neurological symptoms, one or more biological parameters, one or more symptoms determined based on one or more physiological responses from the subject, and any combination thereof.

17. The system according to claim 16, wherein the one or more physiological responses include at least one of the following: one or more eye movements, one or more facial landmarks, one or more body joint positions, one or more pupil movements, one or more speech patterns, and any combination thereof.

18. The system according to claim 16, wherein the one or more symptoms include at least one of the following: dysarthria, aphasia, facial paralysis, gaze deficit, nystagmus, body joint weakness, hemiparesis, ataxia, dyssynergia, dysmetria, and any combination thereof.

19. The system according to claim 16, wherein the one or more biological parameters include at least one of the following: an electrocardiogram, an electroencephalogram, a blood pressure, a pulse, and any combination thereof.

20. The system according to claim 13, wherein the type of the at least one neurological event includes at least one of the following: an acute stroke, an ischemic stroke, a hemorrhagic stroke, a transient ischemic attack, a warning stroke, a mini-stroke, and any combination thereof.

21-36. (canceled)
